(19) United States
(12) Patent Application Publication
(10) Pub. No.: US 2011/0195468 A1
(43) Pub. Date: Aug. 11, 2011

(54) SYSTEM AND PROCESS FOR SEPARATING PURE CHEMICALS FROM BIOMASS EXTRACT

(75) Inventors: Theodora Retsina, Atlanta, GA (US); Vesa Pylkkanen, Atlanta, GA (US)

(73) Assignee: American Process, Inc., Atlanta, GA (US)

(21) Appl. No.: 13/026,273

(22) Filed: Feb. 13, 2011

Related U.S. Application Data
(63) Continuation of application No. PCT/US10/33662, filed on May 5, 2010.
(60) Provisional application No. 61/175,588, filed on May 5, 2009.

(51) Int. Cl. C12P 7/40; C12M 1/40; C12P 7/02; C12P 7/16; C12P 7/10; B01D 35/00

(52) U.S. Cl. 435/136; 435/297.1; 435/155; 435/160; 435/165; 210/259

(57) ABSTRACT

A system or plant and method for the production of pure alcohol, acetic acid or its derivatives from the extract containing hemicelluloses filtered after extraction of woody biomass or directly extracted from woody biomass. The process can be integrated with the host plant process to minimize the effect of loss of heat value from the extracted hemicelluloses and reduce the loading to the effluent plant.

### PRIORITY CROSS REFERENCE

This is a continuation of U.S. provisional patent application No. 61/175,588

### DESCRIPTION

### Field of the Invention

This invention relates, in general, to the post treatment of wood extracts from forest products plants. This treatment specifically converts and separates the soluble fraction of extracted woody material to industrial grade alcohol, alkaline acetate and water.

### BACKGROUND

Forest products industry effluents contain dissolved or mechanically separated wood extract components. The major wood components are lignin, hemicelluloses and cellulose. The current pulping processes preferably separate the lignin with some loss of hemicelluloses. Dissolved lignin and hemicelluloses are burned for process energy and chemical recovery in the most pulping processes. Some or all dissolved wood components from the processes end up in the effluent treatment plant. The recovery, separation, and upgrade of the degraded hemicelluloses into chemicals and derivatives are not practiced. Most common treatment consists of activated sludge wastewater treatment from which the sludge is land filled or burned.

Specifically, the steam explosion process dissolves predominantly hemicelluloses in temperatures above 160 degrees C. Hemicelluloses removed in this process is termed “extract”. The wood chips are released through a pressure reducing valve, commonly termed “blow valve” and are used in the production of medium and hard density board. A concentration of the extract through evaporation is energy intensive, although it is currently practiced to produce molasses.

Previous research indicates that ethanol, acetic acid and their byproducts can be derived from the wood extract. Especially, predominantly hardwood, produces an extract rich in acetic acid and sugars as taught by Amidon et al. in (U.S. Patent Application No. 2007/0079944 A1, Apr. 12, 2007).

Reverse osmosis membranes achieve only 40% rejection of acetic acid according to Perry's Chemical Engineering Handbook (6th ed. p. 17-26). However, 98% rejection of sodium acetate was reported by the same source. Bartels et al. (U.S. Pat. No. 5,028,336, Jul. 2, 1991) discloses alkalizing water-soluble organic acids and removing them by nanofiltration to reduce aqueous effluent dissolved organic solids. No attempts to purify the retentate were reported.

Nothing in the prior art teaches the process to convert acetyl groups to acetic acid in the hydrolysate, evaporate and recover pure alkaline acetate using reverse osmosis membrane. The present application discloses, amongst other things, a process wherein the hemicelluloses in the wood extract can be converted to chemical products in an energy efficient process.

### SUMMARY

The present disclosure relates to, inter alia, a process for the production of alcohol and acetic acid derivatives from wood extract. Treatment of hemicelluloses in the extract through hydrolysis, evaporation, reverse osmosis, fermentation and distillation steps is used to recover and concentrate purified water, alcohol and acetate products. The process may be integrated with the host plant to reuse water and minimize process energy and water consumption.

### BRIEF DESCRIPTION OF THE DRAWING

A more complete understanding of the present invention may be obtained by reference to the following detailed description when read in conjunction with the accompanying drawing wherein:

FIG. 1. illustrates a typical general arrangement of the unit/system/plant operations for wood extract from a steam explosion process. Other wood extract streams are possible. It is a flow diagram example of the invention process. Process steps may be in other sequences and steps may be omitted.

### DETAILED DESCRIPTION OF THE INVENTION

This disclosure is of a system, plant for production and a method. The disclosure below is directed primarily to methods of carrying out the invention, but the methods also encompass a system or plant for carrying out the method.

Wood chips are charged in a batch or continuous reactor vessel, commonly termed “digester” together with steam or hot water and heated to a pressure of 5 to 30 atmospheres to treat the wood chips. In some digesters extract from the wood is removed during this treatment process. The treated wood chips are drained or blown through a valve commonly termed “blow valve” then washed with water to recover the majority of dissolved wood components into the wash filtrate; alternatively dilute wash filtrate may be used in lieu of water. The extract and the wash filtrate are collectively termed “wood extract”. The remaining wood chips are subjected to a manufacturing process, where the wood chips are converted to the final product.

The wood extract contain dissolved xylan, glucan, mannan, arbinan, galactan and acetyl groups in oligomers of hemicelluloses as well as lignin. The wood extract has low organic solids concentration of 0.1% to 12% or more. The majority of water must be removed before an economic treatment of hemicelluloses is possible.

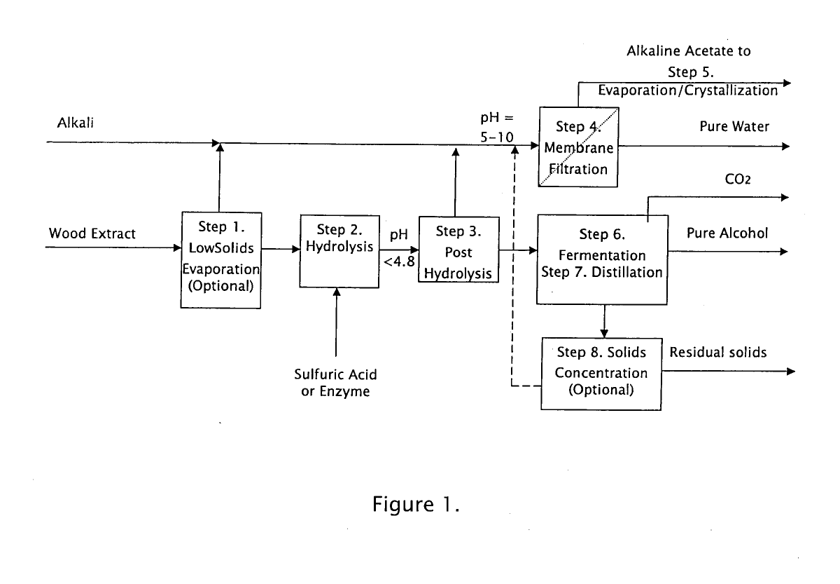

A possible first step of the process is low solids evaporation. FIG. 1 Step 1. The wood extract is concentrated preferably by evaporation, preferably using mechanical vapor recompression evaporation, to a concentration of 1% to 25% or more. If the wood extract initial concentration is over approximately 5%, this step may be omitted. When the pH is below the acetic acid dissociation point of pH 4.8, some acetic acid is split to the evaporator condensate. Under the appropriate economic criteria, this first step could be done with steam evaporation.

A second step of the process is hydrolysis. FIG. 1 Step 2. A mineral acid, preferably sulfuric acid, or enzymes is used to hydrolyze the sugars in the concentrated wood extract from the low solids evaporation step 1. Oligomer hemicelluloses are converted into monomer sugars and acetyl groups are released. The pH of the hydrolyzate from hydrolysis is controlled to maintain acetic acid in unassociated form.

A third step of the process is post hydrolysis evaporation. FIG. 1 Step 3. Hydrolyzate from step 2 is concentrated by evaporation, preferably using mechanical vapor recompression evaporation, up to 25% solids. More of the remaining acetic acid and water is evaporated in this step. Under the appropriate economic criteria, this third step could be done with steam evaporation.

A fourth step of the process is membrane filtration. FIG. 1 Step 4. Hydroxide, carbonate or bicarbonate of sodium, potassium, calcium or magnesium is added to evaporation condensates from steps 1 and 3 to convert acetic acid in the condensates to acetate. The pH of the solution should be such that nearly all acetate ions are associated, but preferably between pH 5 and 10. Acetate associated with such element produces a membrane impermeable acetate salt that is filterable in a membrane, preferably reverse osmosis membrane, with high efficiency. Because the combined condensate from evaporation contains very little impurities, the membrane permeate is a high degree of recovery as high quality water suitable for example as boiler feed water.

A fifth step of the process is acetate concentration. FIG. 1 Step 5. The retentate from the membrane in step 4 is concentrated by evaporation, preferably using mechanical vapor recompression evaporation, up to 50% solids. An industry standard finisher or crystallizer can be used to further concentrate to saleable form as may be required by the market.

A sixth step of the process is fermentation of wood sugars. FIG. 1 Step 6. Sugars in the concentrated hydrolyzate from step 3 post hydrolysis evaporation are fermented in continuous or batch tanks with one or more micro-organisms capable of converting five and six carbon sugars into alcohol and carbon dioxide. The majority of acetic acid, which may inhibit fermentation, was removed in the previous evaporation steps 1 and 3. Some additional acetic acid may be formed during fermentation. Nutrients and pH adjustment chemicals as well as make-up fermentative organism are added in the fermenters as and if needed. Carbon dioxide is removed from the fermenters and scrubbed with cool water for alcohol recovery and the purified gas can be further compressed and sold as industrial grade carbon dioxide. The fermentation broth, commonly termed “beer”, from the fermentation step is sent to step 7, distillation.

A seventh step of the process is distillation of alcohol. FIG. 1 Step 7. The beer from the step 6 fermentation is sent to a beer distillation column to separate the ethanol from the solids and residual sugars. Alcohol leaving as the overhead from the distillation column is recovered at approximately 50 mass-% strength. The final concentration of the alcohol product is performed in a rectifying column and drying system, preferably a molecular sieve, to obtain over 99 mass-% alcohol.

An eighth step of the process is the solids concentration from the stillage. FIG. 1 Step 8. The solids, commonly termed “stillage” from the beer distillation column bottom in step 7 can be further evaporated in an optional concentrator, preferably a mechanical vapor recompression-concentrator, to achieve zero-liquid discharge operation. If the sludge from the optional concentrator is burned, the process may become self-sufficient in its thermal energy needs. The condensate from this step is returned to the reverse osmosis feed in step 4.

It will be appreciated that a combination of all or any of the steps in considered part of this invention and steps may be omitted and still constitute an invention. In the preferred embodiment all disclosed steps are employed.

Integration of the biorefinery with the host forest products plant.

An energy integration analysis of the proposed process indicated that utilizing mechanical vapor recompression evaporators achieves the minimum need for cooling water. The heat generated in the process is absorbed into the product water stream, which can be utilized in the host forest products plant. Furthermore, the reverse osmosis water from step 4 is pure enough to be used in the boiler feed water makeup. This results in a reduction of the energy used in the water heating in the host forest products plant as well as unloading its waste water treatment plant operation.

The claims below form part of this disclosure and are incorporated into the detailed description without repeating the text.

The description of the invention and its applications as set forth herein is illustrative and is not intended to limit the scope of the invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1-30. (canceled)

31. A process for producing an alcohol and an acetate from a biomass-derived extract, said process comprising:
(a) providing a liquid extract produced during cooking of biomass, wherein said liquid extract comprises hemicelluloses and lignin;
(b) if said liquid extract contains a first solids concentration of about 5 wt % or less, concentrating said liquid extract in a first evaporation stage, to produce a concentrated extract, wherein a first amount of acetic acid is evaporated and recovered from said first evaporation stage;
(c) hydrolyzing said liquid extract from step (a) or said concentrated extract from step (b) in the presence of a mineral acid or enzymes, to produce a hydrolyzate comprising monomer sugars and released acetyl groups;
(d) introducing said hydrolyzate to a second evaporation stage, operated at a pH less than 4.8, wherein a second amount of acetic acid is evaporated and recovered from said second evaporation stage;
(e) combining said first and second amounts of acetic acid with an alkali, at a pH selected from 5 to 10, to convert acetic acid to an acetate salt;
(f) filtering out said acetate salt with a membrane; and
(g) fermenting said monomer sugars to an alcohol.

32. The process of claim 31, wherein said cooking of biomass comprises steam cooking, steam explosion, or hot-water cooking.

33. The process of claim 31, wherein said second evaporation stage concentrates said hydrolyzate to a second solids concentration up to about 25 wt %.

34. The process of claim 31, wherein mechanical-vapor recompression is utilized in said first evaporation stage, said second evaporation stage, or both first and second evaporation stages.

35. The process of claim 31, wherein said alkali is selected from the group consisting of sodium hydroxide, potassium hydroxide, calcium hydroxide, magnesium hydroxide, sodium carbonate, potassium carbonate, calcium carbonate, magnesium carbonate, sodium bicarbonate, potassium bicarbonate, calcium bicarbonate, and magnesium bicarbonate.

36. The process of claim 31, wherein said membrane is a reverse-osmosis membrane with a reverse-osmosis concentrate comprising said acetate salt and a reverse-osmosis permeate comprising water.

37. The process of claim 31, said process further comprising concentrating or crystallizing said acetate salt.

38. The process of claim 31, wherein said acetate salt is selected from the group consisting of sodium acetate, potassium acetate, calcium acetate, and magnesium acetate.

39. The process of claim 31, wherein said alcohol is ethanol or butanol.

40. The process of claim 31, said process further comprising distilling and drying said alcohol to produce a purified alcohol and a distillation bottoms stream.

41. The process of claim 40, said process further comprising introducing said distillation bottoms stream to a third evaporation stage, to produce residual solids.

42. The process of claim 41, wherein a liquid stream from said third evaporation stage is recycled to said membrane.

43. The process of claim 31, wherein said process is a zero-liquid-discharge process.

44. The process of claim 31, wherein said liquid extract is produced prior to, or during, a biomass process selected from the group consisting of a pulping process, a board process, and a combustion process.

45. A zero-liquid-discharge process for producing an alcohol, an acetate, clean water, carbon dioxide, and residual solids from a biomass-derived extract, said process comprising:
(a) providing a liquid extract produced during steam cooking or hot-water cooking of biomass, wherein said liquid extract comprises hemicelluloses and lignin;
(b) hydrolyzing said liquid extract in the presence of a mineral acid or enzymes, to produce a hydrolyzate comprising monomer sugars and released acetyl groups;
(c) concentrating said hydrolyzate in a first evaporation stage, to produce a concentrated hydrolyzate with a solids concentration of about 5 wt % to about 25 wt %, wherein said first evaporation stage is operated at a pH less than 4.8 to maintain acetic acid in unassociated form, whereby at least a portion of said acetic acid is evaporated from said first evaporation stage to form evaporated acetic acid;
(d) combining said evaporated acetic acid with an alkali, at a pH selected from 5 to 10, to convert said evaporated acetic acid to an acetate salt, wherein said acetate salt is selected from the group consisting of sodium acetate, potassium acetate, calcium acetate, and magnesium acetate;
(e) filtering out said acetate salt with a reverse-osmosis membrane, to produce a reverse-osmosis concentrate comprising said acetate salt and a reverse-osmosis permeate comprising clean water;
(f) concentrating or crystallizing said acetate salt, to produce a purified acetate salt;
(g) fermenting said monomer sugars to an alcohol and carbon dioxide, wherein said alcohol is ethanol or butanol;
(h) distilling said alcohol to produce a purified alcohol and a distillation bottoms stream;
(i) introducing said distillation bottoms stream to a second evaporation stage, to produce residual solids; and
(j) recycling condensate from said second evaporation stage to said reverse osmosis membrane.

46. A system to recover acetic acid from a liquid solution, said system comprising:
(a) an input stream comprising a liquid solution including acetic acid;
(b) a first evaporation unit, in communication with said input stream, for vaporizing a first amount of acetic acid contained in said liquid solution, wherein said first evaporation unit contains a liquid phase controlled to a pH below 4.8;
(c) a first output vapor stream and first output liquid stream of said first evaporation unit;
(d) a second evaporation unit downstream of said first evaporation unit, for vaporizing a second amount of acetic acid contained in said first output liquid stream of said first evaporation unit, wherein said second evaporation unit contains a liquid phase controlled to a pH below 4.8;
(e) a second output vapor stream and second output liquid stream of said second evaporation unit;
(f) an alkali input stream comprising an alkali, wherein said alkali input stream is in communication with said first output vapor stream and said second output vapor stream, or condensates thereof, for converting at least some of said first and second amounts of acetic acid to alkaline acetate at a pH controlled from 5 to 10; and
(g) a membrane for filtering out said alkaline acetate in a membrane retentate.

47. The system of claim 46, wherein said alkaline acetate is selected from the group consisting of sodium acetate, potassium acetate, calcium acetate, and magnesium acetate.

48. The system of claim 46, said system further comprising a mechanical-vapor recompression evaporator, a crystallizer, or both of these, to concentrate said alkaline acetate.

49. The system of claim 46, said system further comprising a reactor disposed between said first evaporation unit and said second evaporation unit, wherein said reactor generates additional acetic acid.

50. The system of claim 49, wherein said reactor is a hydrolysis reactor operated with a mineral acid or enzymes for hydrolyzing biomass sugar oligomers to monomers and to said additional acetic acid.
